(19) United States
(12) Patent Application Publication
(10) Pub. No.: US 2026/0063620 A1
(43) Pub. Date: Mar. 5, 2026

(54) METHOD FOR MEASURING EXTENT OF MICROPLASTIC BIOACCUMULATION IN BIVALVES IN EXTREME DEEP-SEA ENVIRONMENTS

(71) Applicant: SOUTHERN MARINE SCIENCE AND ENGINEERING GUANGDONG LABORATORY (GUANGZHOU), Guangzhou (CN)

(72) Inventors: Jingchun FENG, Guangzhou (CN); Canrong LI, Guangzhou (CN); Si ZHANG, Guangzhou (CN); Zhifeng YANG, Guangzhou (CN)

(73) Assignee: SOUTHERN MARINE SCIENCE AND ENGINEERING GUANGDONG LABORATORY (GUANGZHOU), Guangzhou (CN)

(21) Appl. No.: 19/312,383

(22) Filed: Aug. 28, 2025

(30) Foreign Application Priority Data
Aug. 28, 2024 (CN) 202411192032.6

(51) Int. Cl. G01N 33/50; C12Q 1/37; G01N 1/40; G01N 1/42

(52) U.S. Cl. CPC G01N 33/50; C12Q 1/37; G01N 1/4077; G01N 1/42; G01N 2001/4088

(57) ABSTRACT

A method for detecting the extent of microplastic accumulation in bivalve organisms in extreme deep-sea environments is provided. This method is performed through sampling of biological communities to represent the community structure of that biological bed layer, then morphological characterization statistics are conducted to classify the age stage data of individual bivalves, multivariate factor analysis is used to obtain the individual bivalves with the greatest degree of contribution in each classified age stage, subsequently tissue-specific microplastic extraction is performed, the morphology of seafloor microplastics is restored and streamlined identification of full-size microplastics is considered, carbon-14 dating is used to trace the duration of microplastic adsorption by each individual bivalve, dynamic accumulation curves for seafloor bivalves are constructed by connecting each bivalve's survival duration, and finally this is scaled up to the entire bivalve bed to derive the microplastic accumulation rate and historical accumulation of the entire extreme ecosystem.

### TECHNICAL FIELD

The present application relates to the technical fields of ecological and biophysiological investigations of extreme deep-sea environments, extraction and detection of novel pollutants, and migration and transformation of novel pollutants in vivo, and in particular to a method for measuring the extent of microplastic bioaccumulation in bivalves in extreme deep-sea environments.

### BACKGROUND TECHNOLOGY

Deep-sea extreme environments refer to oceanic extreme environment areas such as seamounts, hydrothermal vents, and cold seeps located at water depths below 1000 m, characterized by darkness, high pressure, low and stable temperatures, and relatively scarce nutrients. Deep-sea methane seeps are a special type within deep-sea extreme environments. The formation of these areas comes from changes in seafloor geological topography or decomposition of natural gas hydrates in sediments, where methane seep fluids rich in methane and hydrogen sulfide gush out from cracks in seafloor sediments, forming special chemosynthetic ecosystems that use methane as a carbon source. In this extreme environment, methane seep microbial communities form symbiotic relationships with bivalves (such as mussels, white clams, and vesicomyids). Most of these microorganisms coexist in the gills of bivalves, utilizing nutrients released from seafloor cracks for chemosynthesis to provide organic matter to the bivalves, while the corresponding bivalves obtain particles containing microorganisms by filtering seawater. These bivalves can not only survive in methane seeps but also rely on compounds such as methane and hydrogen sulfide as energy sources, forming unique ecological niches. When studying the age of deep-sea organisms or organic matter, researchers typically use carbon-14 dating methods to determine their growth history, utilizing the decay patterns of radioactive isotopes to determine age, which has high precision and reliability. Bivalves, due to their seafloor and filter-feeding lifestyle and their chitin shells with adsorptive capacity, are more likely to accumulate pollutants from water compared to other species. Therefore, marine bivalves, especially mussels, are commonly used as model organisms in research monitoring marine pollution conditions and the absorption and effects of emerging pollutants.

Microplastics are a type of emerging environmental pollutant, mainly referring to plastic particles with diameters smaller than 5 mm, which have attracted global attention due to their widespread occurrence worldwide. More than 10 million tons of plastic enter the ocean annually, forming large amounts of microplastics through long-term weathering, photolysis, and biodegradation, posing serious threats to marine environments and marine organisms. The impact of microplastics on marine organisms varies among species, with seafloor filter-feeding organisms being more sensitive to microplastics and capable of ingesting more microplastics. According to current laboratory simulation studies on bivalve mussels, microplastics commonly accumulate in the gills, digestive tract, muscle tissue, gonads, adductor muscles, and foot within mussels, with the digestive tract being the primary accumulation site. When microplastics enter the mussel's digestive tract, they undergo a series of physicochemical changes including fragmentation, decomposition, and possible biodegradation. However, due to their very slow degradation process, most microplastics in mussels maintain their original morphology and structure. Therefore, bivalves in methane seeps have enormous potential for microplastic accumulation and can reflect the long-term history of microplastic pollution in seafloor extreme environments. Bivalves serve as biological indicators of methane seep activity intensity in seafloor areas and can actively absorb surrounding seawater microplastics through feeding activities. By monitoring microplastic content in their living bodies and observing shell sizes, it is possible to assess the distribution, migration, and ecological impact of microplastics in marine extreme environments, indirectly reflecting the degree of microplastic absorption by deep-sea ecosystems and the environmental behavior of microplastics.

Currently, in the field of characterizing the degree of ecosystem microplastic absorption, domestic and international scholars mainly focus on studying microplastics present in organisms within estuarine and intertidal ecosystems, with no reference cases for related research on indicator organisms in deep-sea extreme environments, especially in methane seeps. However, deep-sea extreme environments, as important components of Earth's ecosystems, have significant importance for assessing the pollution status of the entire marine environment through the microplastic content and distribution in their organisms. By studying the dynamic enrichment process of microplastics in deep-sea organisms and quantifying the temporal scale of deep-sea microplastic pollution acceptance since the beginning of plastic production, we can reveal the distribution, migration, and transformation patterns of microplastics in marine extreme environments, providing a scientific basis for formulating effective pollution prevention and control measures.

Through reviewing extensive literature on the degree of microplastic absorption by ecosystems and extraction of microplastics from aquatic organisms, there is a lack of any patents involving the characterization of the long-term microplastic accumulation capacity of entire ecosystems. Most scholars extract microplastics contained in single-medium environments (such as water/sediment/organisms) and simply calculate pollution risk assessment indices for microplastics to obtain their pollution status on the ecological environment. However, this cannot withstand the testing of sampling randomness and temporal limitations, and the results obtained do not represent the ecological environment as a whole. Experimental extraction of microplastic adsorption by regional indicator organisms, combined with theoretical accumulation verification methods, is the core requirement for steadily exploring the rate of microplastic adsorption by ecosystems. The current conventional method for extracting microplastic content from bivalves involves first using automated mechanical equipment and handheld tools to collect organisms and perform dissection and sampling, then conducting indoor microplastic extraction and detection. The extraction process includes grinding biological tissues to obtain homogenate, using alkaline digestion methods to release microplastics, and separating microplastic particles through density flotation and vacuum filtration methods. The detection process mainly uses

Fourier transform infrared spectroscopy, Raman spectroscopy, and other detection instruments to determine the types and abundance of microplastics. Currently, existing methods for extracting microplastics from bivalves have the following disadvantages: \textcircled{1} The microplastic extraction process excessively alters the morphology and abundance of microplastics rather than preserving their original state. During preprocessing, most patents commonly obtain tissue homogenate through grinding to improve the efficiency of organic matter digestion, but this method destroys the morphology of fibrous microplastics in organisms, affecting subsequent statistical results of external characterization of microplastics in organisms. In the digestion stage, whether using acid-base digestion or strong oxidant digestion methods, individual use will dissolve specific types of microplastics, causing the in-situ microplastic absorption abundance obtained from experiments to deviate significantly from actual values. In extraction and separation experiments, density flotation methods are usually used to separate microplastics, but commonly used flotation agents all have limitations and cannot meet the requirements of all experiments. \textcircled{2} Focus on microplastic occurrence results in single-medium carriers while ignoring the absorption capacity of entire extreme ecosystems. Most patents focus on simple analysis of extracted microplastic abundance within water body/water area/sediment media, only judging the current pollution status of water areas/sediment layers by counting microplastic types, abundance, morphology, etc., and calculating ecological risk indices, lacking technical methods to assess the capacity of entire environments to carry microplastics and the dynamic enrichment process of microplastics from an ecosystem perspective. \textcircled{3} Emphasis on static instantaneous microplastic pollution conditions while lacking dynamic enrichment analysis of regionalized microplastic colonization on a temporal scale. Currently available regional microplastic risk assessment methods mainly study the microplastic abundance contained in single-medium environments at the time and conduct localized time-limited assessments, without involving any characterization means for the dynamic accumulation capacity of microplastics on a temporal scale from their birth through transport to the deep seafloor, and even long-term dynamic accumulation by marine extreme ecosystems.

### CONTENT OF THE INVENTION

The present application solves multiple defects existing in the prior art for seafloor bivalve microplastic extraction experiments, the lack of characterization capability for ecosystem spatial microplastic adsorption, and the absence of long-term dynamic microplastic accumulation assessment in organisms. The present application proposes a method for measuring the extent of microplastic bioaccumulation in bivalves in extreme deep-sea environments. The method proposed by the present application conducts a series of adaptive optimizations in community survey methods for capturing seafloor indicator organisms, multivariate factor analysis to find bivalves with community characteristics, bivalve microplastic enrichment experiments, and construction of dynamic adsorption curves for ecosystem microplastic capacity to intuitively demonstrate deep-sea microplastic pollution impacts. The objective is to select typical age-group bivalves based on typical methane seep extreme environments on the seafloor and classify them according to bivalve morphological characteristics for microplastic extraction experiments, analyze the principles by which individual bivalves serve as community surrogates in methane seeps, and improve fundamental research on the degree of microplastic accumulation in indicator organisms of extreme ecosystems.

The objective of the present application is to provide a method for measuring the extent of microplastic bioaccumulation in bivalves in extreme deep-sea environments, comprising the following steps:
S1. Collect and estimate the total number of bivalves in the area:
S11. Search for living bivalve beds in the early and middle development stages of methane seeps in the deep-sea methane seeps, measure the bed area, and find the equivalent diameter and geometric center of the bed area;
S12. Capture the bivalve bed several times, with the capture positions located at two ends and the midpoint of the equivalent circle radius; after capture and retrieval, count the number of living bivalves, wash the bivalves, and store the bivalves frozen after draining the water;
S13. Based on the number of bivalves captured several times, roughly estimate the total number of living bivalves in the entire bivalve bed;
S2. Bivalve morphology measurement: Thaw the captured bivalve samples, perform morphological data measurements and weighing in batches, and divide the bivalves according to increasing shell length into four age groups of juvenile, young, middle-aged, and elderly;
S3. Multivariate factor analysis: based on the obtained morphological data in step S2, associate and analyze the relationship between bivalves of different ages on the seafloor and their external length and wet weight using a multifactor statistical analysis method, and identify the top five individual bivalves representing the characteristics of each of the four age groups, resulting in a total of twenty bivalves;
S4. Microplastic enrichment and extraction: separate and extract microplastics from each tissue of the representative bivalve samples of the four age groups selected by multivariate factor analysis in step S3, ultimately obtaining a purified microplastic filter membrane for convenient subsequent observation and detection;
S5. Instrument identification: Sequentially identify microplastics with sizes ranging from 0.001-5 mm in the purified microplastic filter membrane obtained in step S4, and comprehensively analyze the occurrence state of microplastics within bivalve bodies and the physical changes of microplastics after entering the deep sea;
S6. Carbon-14 dating method: Use carbon-14 dating to determine the chronological ages of corresponding individual bivalves, thereby converting the lifespan duration of the individual bivalves from the four age groups into time periods of microplastic absorption by seafloor bivalves;
S7. Dynamic microplastic accumulation model for bivalves: Use an adsorption kinetic model to construct microplastic accumulation curves within bivalve bodies; calculate accumulation coefficients to represent the degree of microplastic accumulation in sample bodies relative to the environmental concentration; based on the microplastic concentration in the top five individual bivalves representing the group characteristics selected from the four age groups and their corresponding carbon-14 estimated ages, jointly establish a time-series model; extrapolate the temporal microplastic adsorption patterns of the individual bivalves obtained from all measurement data to the entire bivalve bed through sampling patterns, and approximately deduce the rate of microplastic accumulation and total microplastic accumulation in the entire seafloor extreme environment ecosystem.

The method proposed by the present application obtains bivalves from each age group that can substitute for entire community characteristics through multivariate factor analysis, selects typical bivalves from each age group, then employs a microplastic extraction protocol without homogenization, with mixed solution digestion, and eliminating density flotation operations to clarify the adsorption abundance of microplastics in extreme environment seawater by indicator organisms. Finally, carbon-14 dating is used to determine the actual survival duration of selected bivalves, with the purpose of utilizing instantaneous microplastic concentrations accumulated in bivalves of each age group in methane seeps, using area approximation estimation methods to convert them into dynamic microplastic accumulation curves on a centennial scale for extreme ecosystems, laying the foundation for further research on deep-sea microplastic migration and transformation processes and their impacts on organisms.

The present application combines carbon-14 dating with obtained microplastic concentrations within bivalve bodies to construct microplastic accumulation curves over the full lifespan of bivalves to reveal the long-term dynamic microplastic adsorption capacity of seafloor extreme ecosystems. This is the first integrated analysis protocol to measure the microplastic uptake content in methane seep ecosystems using time and space as entry points.

In step S1, the area approximation estimation method is used to roughly estimate the total amount of living bivalves in the entire mussel bed. The specific steps are: When conducting resource exploration in deep-sea methane seeps using a remotely operated vehicle (ROV), locate living bivalve biological cluster beds in the early to middle stages of cold seep development, then use a multibeam bathymetry system to measure the seafloor living bivalve bed area (A). Based on the area equivalence principle, find its equivalent diameter (D, as in equation (1)) and calculate its geometric center using integration methods, record the GPS coordinates, and set the grab sampling area of the deployed television grab as A_{grab}. Use the Television grab with fixed grab area to sample the bivalve bed several times (described below using 3 times as an example), with sample positions located at both ends of the equivalent circle radius and at the midpoint. After bringing samples to the surface, count the number of living bivalves carried in each sample, use cruise water equipment to wash mud from bivalve surfaces, remove shell remains without soft tissue, drain water, and individually bag and record the serial numbers. Bivalve juveniles with shell length below 10 mm are not included in the statistical range and are stored in a −20° C. horizontal freezer. The cumulative count of living bivalves from the three samples is set as N₁, N₂, N₃. By introducing the cluster coefficient of living bivalves (C, the ratio of density variance to the square of average density, as in equation (2)), roughly estimate the total number of bivalves (Nₜₒₜₐₗ) of the entire living bivalve bed, as shown in equation (3) below:

$$D = 2 \sqrt{\frac{A}{π}} \qquad ( 1 )$$

$$\overline{ρ} = \frac{{N}_{1} + {N}_{2} + {N}_{3}}{3 {A}_{g r a b}} , \\ C = \frac{{( {N}_{1} / {A}_{g r a b} - \overline{ρ} )}^{2} + {( {N}_{2} / {A}_{g r a b} - \overline{ρ} )}^{2} + {( {N}_{3} / {A}_{g r a b} - \overline{ρ} )}^{2}}{3 {\overline{ρ}}^{2}} \qquad ( 2 )$$

$${N}_{t otal} = \frac{{N}_{1} + {N}_{2} + {N}_{3}}{3} \times \frac{A}{{A}_{g r a b}} \times C \qquad ( 3 )$$

The present application is the first to numerically characterize the microplastic accumulation ability of deep-sea extreme ecosystems, breaking through the constraints of current static microplastic pollution. Using the bivalve's own survival age as an entry point, several time sections are connected into dynamic microplastic accumulation curves throughout the entire bivalve lifecycle, creating a precedent for the expression of microplastic accumulation rates in ecosystems and filling gaps in this field. Deep-sea bivalves are typical indicator animals for microplastics, and investigating the long-term dynamic accumulation of microplastics within their bodies is of great significance for assessing microplastic pollution levels in extreme deep-sea environments, dynamically identifying the degree of microplastic accumulation in extreme environments on a centennial scale, and understanding microplastic migration and transformation processes in the ocean.

The method proposed by the present application first uses area approximation estimation for rational sampling collection of biological communities under extreme environments, maximizing representation of the community structure of the biological bed layer, then conducts bivalve morphological characterization statistics to simplify classification of age stage data for each individual bivalve. Multivariate factor analysis is used to obtain the top five individual bivalves with the greatest contribution and best representation of the entire age community from each classified age stage. Subsequently, microplastic extraction optimization experiments are conducted on separate tissues to restore the morphology of seafloor microplastics while considering streamlined identification protocols for full-scale microplastics. Carbon-14 dating is used to trace the duration of microplastic adsorption by each individual bivalve, constructing dynamic accumulation curves for seafloor bivalves by connecting each bivalve's survival duration, and finally scaling up to the entire bivalve bed to derive the accumulation rate and historical accumulation of microplastics in the entire extreme ecosystem. The present application aims to describe the microplastic accumulation rate of specific cold seep indicator organisms in extreme ecosystems represented by methane seeps and approximately assess the temporal accumulation capacity of the entire seafloor system for microplastics since the beginning of plastic production. This is a novel approach for assessing microplastic pollution that few have proposed, providing an alternative perspective for quantifying the damaging effects of human activities on extreme ecosystems, indirectly demonstrating the historical accumulation of microplastics in ecosystems and enabling future pollution trend predictions, providing a scientific basis and technical support for formulating effective marine environmental protection strategies and clarifying marine microplastic source-sink relationships.

Preferably, the steps of step S2 specifically are: perform morphological measurements and weighing in batches for all of the bivalve samples sampled several times from the seafloor; measure the three body length dimensions of the bivalves to obtain a shell length L, shell width W, and shell height H for each live bivalve; based on general body length standards corresponding to different age groups of bivalves, establish four shell length ranges, which are 0<L<55 mm, 55 mm<L<80 mm, 80 mm<L<110 mm, 110 mm<L, corresponding to the four age groups classified as juvenile, young, middle-aged, and elderly.

Step S2 aims to integrate the basic morphological data of each bivalve, categorizing all collected bivalve samples into four age groups: juvenile, young, middle-aged, and elderly. All microplastics adsorbed or ingested by each mussel from birth to survival until the moment of sampling will either remain within the body or be partially excreted. This approach uses reorganized time sections to indirectly characterize the state of microplastic accumulation experienced by this local extreme ecosystem during each growth cycle, connecting them into a dynamic microplastic adsorption curve for the methane seep over nearly a century.

Preferably, the steps of step S3 specifically are:
S31. Based on the morphological data obtained in step S2, perform categorical sampling, add a new age classification array to the original four columns of basic data, replace the age groups of juvenile, young, middle-aged, elderly with numbers 1, 2, 3, 4, set the age groups as a categorical variable array, and set the other four columns of morphological arrays as quantitative variable arrays;
S32. Create an indicator matrix for the existing age classification array, calculate its standardized contingency table, and perform singular value decomposition; normalize the first singular value according to the obtained diagonal matrix elements, and expand the age classification array into two-dimensional space for display to obtain the MCA standardized coordinate matrix;
S33. Perform normalization or centering standardization on the remaining four columns of quantitative arrays, calculate their covariance matrix, and obtain their eigenvalues and eigenvectors; select the first k eigenvectors to form matrix P, and project into a new two-dimensional space to obtain the coordinate matrix after dimensionality reduction, and from this, calculate the factor score coefficient matrix of each quantitative array after dimensionality reduction;
S34. After being normalized or centered, divide the four columns of quantitative arrays by the square root of the eigenvalue of the first axis of their covariance matrix to obtain the factor loading matrix describing the linear combination relationships between the four quantitative arrays and the common factors, and merge this loading matrix with the first singular value standardized matrix of the age classification array to form a global factor loading T matrix;
S35. Perform multivariate factor analysis on the global factor loading T matrix, and convert matrix TTT into projection matrix P; project matrix T into the multivariate factor analysis model ranking chart through matrix P to calculate the variable load contribution rate of the five morphological arrays to the global factor coordinate matrix;
S36. Rank the variable load contribution rate of each array, and select the top five mussels of each age group as representatives of microplastic accumulation characteristics of the community in the corresponding age group.

Step S3 conducts categorical sampling based on the basic morphological data obtained in step S2. A new age classification column is added to the original four arrays of basic data, using numbers 1, 2, 3, 4 to respectively represent the full age stages of each bivalve-juvenile, young, middle-aged, elderly. Subsequently, various statistical methods are used to determine which individual bivalves have the highest contribution rate to shell length indicators under the four age groupings, and which can best represent the characteristic information of the corresponding individual age group. For example, using multivariate factor analysis (MFA) as the selected statistical method, first determine the nature of each variable (categorical or quantitative) in the 5 data columns, then standardize each variable column and perform eigen decomposition, execute multiple correspondence analysis (MCA) for categorical variable columns, execute principal component analysis (PCA) for quantitative variable columns, project the eigenvalues of each group of variables in the MFA global ordination plot and arrange them according to the contribution rate proportion of each bivalve individual within the four age groups, showing the maximum explanatory degree of each bivalve individual for revealing the variance variation of the community morphological dataset, evaluate which eigenvalues of the variable columns have higher contributions to the MFA ordination space, and determine which types of data columns mainly dominate each dimension of MFA. The objective of this step is to select representative samples from four typical age group bivalve communities, achieving the reflection of microplastic accumulation levels in representative age layer samples through experimental results from a small number of typical samples, greatly reducing the number of required samples for detection and improving research efficiency. The specific steps are as follows:

\textcircled{1} Dimensionally standardize the obtained characteristic data variable columns according to their data attributes. For categorical datasets such as age classification arrays, standardize data through the first singular value of each variable. For this purpose, it is first necessary to create an indicator matrix (Z₀) for the existing age classification data, with dimensions n×4, where n is the number of selected individual bivalves. Each row of the indicator matrix (Z₀) corresponds to an observation value, and each column corresponds to an age category. If the observation value belongs to that category, the corresponding element is 1, otherwise it is 0.

\textcircled{2} Calculate the standardized contingency table (Burt Matrix). The Burt matrix is calculated through the product of the above-obtained indicator matrix (Z₀) and its transpose matrix (Z^{T}), as shown in equation (4).

$$Burt = Z \cdot {Z}^{T} \qquad ( 4 )$$

Wherein, Burt is a 4×4 matrix, matrix element Bᵢⱼ represents the number of times class i and class j appear together in the dataset, and the diagonal elements of the Burt matrix represent the frequency of individual age categories appearing in the dataset.

\textcircled{3} Singular value decomposition. Perform singular value decomposition on the Burt matrix to extract singular values and corresponding singular vectors. The first singular value is the largest among these singular values, capturing the most principal variability in the data. First, perform necessary centering on the obtained Burt matrix to adjust each element's influence on eliminating marginal frequencies. The centering formula is shown in equation (5).

$${B}_{ij}^{C e n t} = {B}_{ij} - \overline{{B}_{i .}} - \overline{{B}_{. j}} - \overline{{B}_{k}} \qquad ( 5 )$$

Wherein Bᵢⱼ is the original element in the Burt matrix, B̅_̅{̅i̅.̅}̅ is the average value of the corresponding i-th row of the matrix, B̅_̅{̅.̅j̅}̅ is the average value of the corresponding j-th column, and B̅ₖ̅ is the overall average value of the entire matrix.

Subsequently, standardize the centered Burt matrix to make the sum of rows and columns equal to 1, reflecting the relative importance of different categories, as detailed in equations (6)-(8).

$${B}_{ij}^{S t a n d} = \frac{{B}_{i j}^{Cent}}{\sqrt{{p}_{i} - {p}_{j}}} \qquad ( 6 )$$

$${p}_{i} = \frac{{\sum }_{j = 1}^{n} {B}_{i j}}{{\sum }_{i = 1}^{n} {\sum }_{j = 1}^{n} {B}_{i j}} \qquad ( 7 )$$

$${p}_{i} = \frac{{\sum }_{i = 1}^{n} {B}_{i j}}{{\sum }_{i = 1}^{n} {\sum }_{j = 1}^{n} {B}_{i j}} \qquad ( 8 )$$

Wherein, pᵢ is the marginal sum of the i-th row divided by the sum of matrix elements, pⱼ is the marginal sum of the j-th column divided by the sum of matrix elements, and n is the dimension of the Burt matrix.

The processed Burt matrix applies singular value decomposition (SVD), as shown in equation (9).

$${B}_{i j}^{S t a n d} = U \sum {V}^{T} \qquad ( 9 )$$

Wherein, the column vectors of U are the projections of each age stage corresponding to individual bivalves on the singular value-defined dimensions, the column vectors of V represent the projections of the four age stages on new dimensions, and we arrange the diagonal elements of the diagonal matrix Σ in descending order and select the first singular value among them.

\textcircled{4} First singular value standardization. Divide the frequency of each age level of each bivalve individual by the first singular value (Σ₁₁). The objective of this step is to reduce the age scale differences existing among different individual bivalves themselves, as shown in equation (10).

$${B}_{i j}^{N o r m} = \frac{{B}_{i j}^{S t a n d}}{{\sum }_{11}} \qquad ( 10 )$$

\textcircled{5} Extract MCA standardized coordinates. The row coordinates and column coordinates of the standardized categorical column vectors can be expressed by the corresponding column data of U, V and Σ/Σ₁₁. Project the age classification array data into 2-dimensional space for visualization, obtaining the interaction range and effect intervals between different morphological variables in age groups, as shown in equations (11)-(12).

$$F = U \cdot \sum / {\sum }_{11} \qquad ( 11 )$$

$$G = V \cdot \sum / {\sum }_{11} \qquad ( 12 )$$

Wherein, F is the row coordinate matrix of age data, and each row Fᵢ represents the standardized abscissa of the i-th age value in Bᵢⱼ^{Norm}; similarly, G is the corresponding column coordinate matrix, and each column Gⱼ represents the standardized column coordinate of the j-th age value in Bᵢⱼ^{Norm}.

\textcircled{6} Subsequently, normalize or center-standardize the remaining 4 quantitative arrays respectively to eliminate the influence of different dimensions between continuous data. The expressions are as follows in equations (13)-(14):

$${Z}_{1} = \frac{x - {x}_{min}}{{x}_{max} - {x}_{min}} \qquad ( 13 )$$

$${Z}_{2} = \frac{x - {x}_{m e a n}}{σ} \qquad ( 14 )$$

Wherein, Z is the processed data vector, x is the input morphological observation value of the individual bivalve, xₘᵢₙ is the minimum value in a single data column, xₘₐₓ is the maximum value in the corresponding data column, xₘₑₐₙ is the average value of the corresponding data column, and σ is the variance of the data column.

\textcircled{7} Calculate the covariance matrix of the normalized or centered quantitative arrays. The covariance matrix describes the degree of correlation between various morphological parameters within the quantitative arrays. For a dataset with n variables, the covariance matrix is an n×n matrix, wherein element (X, Y) represents the covariance between variable X and variable Y, as shown in equations (15) and (16):

$$cov ( X , Y ) = \frac{{\sum }_{i = 1}^{n} ( {X}_{i} - \overline{X} ) ( {Y}_{i} - \overline{Y} )}{n - 1} \qquad ( 15 )$$

$$\overline{X} = \frac{1}{n} {\sum }_{i = 1}^{n} {X}_{i} , \overline{Y} = \frac{1}{n} {\sum }_{i = 1}^{n} {Y}_{i} \qquad ( 16 )$$

Wherein, cov(X,Y) is the covariance matrix between pairwise variables in each quantitative array column, X̅ and Y̅ are the column vector averages between pairwise variables within the quantitative arrays.

\textcircled{8} Calculate the eigenvalues and eigenvectors of the covariance matrix within each quantitative array column. Eigenvalues represent the importance degree of the principal component corresponding to each eigenvector, while eigenvectors represent the direction of that principal component in the original variable space, as shown in equation (17):

$$( A - λ E ) x = 0 \qquad ( 17 )$$

Wherein, A is the covariance matrix obtained from the above equation, λ is the eigenvalue corresponding to the covariance matrix, E is the identity matrix, and x is the eigenvector corresponding to the covariance matrix to be solved.

\textcircled{9} Sort according to the numerical size of λ, select eigenvalues and their corresponding eigenvectors where λ>1. The number k of λ is the first k eigenvectors with the largest eigenvalues selected.

\textcircled{10} Form matrix P with the first k eigenvectors, and project into the new space to obtain a new low-dimensional representation. The projection is shown in equation (18):

$$Z = XP \qquad ( 18 )$$

Wherein, Z is the data matrix after dimensionality reduction, X is the four quantitative data matrices, and P is the matrix composed of the first k eigenvectors.

\textcircled{11} Calculate the factor score coefficient matrix of each dimensionally reduced quantitative data matrix, as shown in equations (19)-(21):

$${σ}_{X} = \sqrt{\frac{{\sum }_{i = 1}^{n} {( {X}_{i} - \overline{X} )}^{2}}{n - 1}} , {σ}_{Y} = \sqrt{\frac{{\sum }_{i = 1}^{n} {( {Y}_{i} - \overline{Y} )}^{2}}{n - 1}} \qquad ( 19 )$$

$${R}_{xy} = cov ( X , Y ) / ( {σ}_{X} \cdot {σ}_{Y} ) \qquad ( 20 )$$

$$A = ZC \qquad ( 21 )$$

Wherein, σ_{X} and σ_{Y} respectively represent the standard deviations of pairwise variables in each quantitative data group, R_{XY} represents the correlation coefficient of pairwise variables in the corresponding array, cov(X,Y) is the covariance matrix of pairwise variables, Z is the data matrix of each quantitative array after dimensionality reduction to k dimensions, C is the correlation coefficient matrix composed of R_{XY} from each array matrix, and A is the factor score coefficient matrix.

\textcircled{12} Calculate the normalized quantitative data groups. Divide the normalized or centered quantitative morphological data groups obtained from step \textcircled{6} above by the singular values of the first PCA axis of each quantitative array found in step \textcircled{8} above, obtaining a matrix describing the linear combination relationship between variables and extracted common factors. The specific calculation is shown in equation (22):

$${T}_{n} = \frac{{Z}_{n}}{{φ}_{1}} \qquad ( 22 )$$

Wherein, Tₙ is the factor loading matrix of the 4 quantitative arrays, Zₙ is the normalized or centered quantitative morphological array, and φ₁ is the singular value of the first PCA axis of each quantitative morphological array (i.e., the square root of the eigenvalue).

\textcircled{13} Contribution normalization. Combine the normalized quantitative data groups obtained from step \textcircled{12} with the age categorical array standardized by the first singular value from step \textcircled{4} to form the global factor loading T matrix.

\textcircled{14} Perform multivariate factor analysis on the global factor loading matrix T to explore the global structure and patterns of the entire bivalve individual array, revealing the global principal components and contribution rates in the age-morphological dataset. This is equivalent to calculating the singular value decomposition of the global factor loading matrix, obtaining individual bivalves that can significantly substitute for the entire data group under the dominance of the shell length array. The calculation is shown in equations (23)-(24):

$$T = U Δ {V}^{T} \qquad ( 23 )$$

$${U}^{T} U = {V}^{T} V = I \qquad ( 24 )$$

Wherein, T is the global factor loading matrix converted from categorical and quantitative arrays, U and V are respectively the left and right singular vectors of matrix T, A is the diagonal matrix of singular values, and I is the identity matrix.

\textcircled{15} Convert the matrix TTT composed of the global factor loading matrix into projection matrix P, satisfying equation (25):

$${M}^{- \frac{1}{2}} U Δ = ( {TT}^{T} ) \times ( {M}^{- \frac{1}{2}} U {Δ}^{- 1} ) = ( {TT}^{T} ) P \qquad ( 25 )$$

Wherein, M is an I×I diagonal matrix, and P is the projection matrix.

\textcircled{16} Use projection matrix P to project the global factor loading matrix onto the multivariate factor analysis model ordination plot, and evaluate the common structure and differences between arrays through object and variable ordination plots to obtain the serial numbers of the typical individual bivalves. The calculation expression (26) is:

$${K}_{n} = D \times ( {T}_{n} {T}_{n}^{T} ) P \qquad ( 26 )$$

Wherein, Kₙ is the two-dimensional coordinate matrix of each bivalve characteristic array subset after projection, D is the number of groups within the bivalve characteristic array, and Tₙ is the column matrix of the global factor loading matrix for each bivalve characteristic array.

Calculate the correlation between the two-dimensional coordinate matrix of each bivalve characteristic array column and the global factor coordinate matrix, forming the variable loading contribution rate R_{nMFA}. Expression (27) is:

$${R}_{nMFA} = \frac{{\sum }_{i} {\sum }_{j} ( {K}_{ij} - \overline{K} ) ( {K}_{nij} - \overline{{K}_{n}} )}{\sqrt{( {\sum }_{i} {\sum }_{j} {( {K}_{ij} - \overline{K} )}^{2} ) ( {\sum }_{i} {\sum }_{j} {( {K}_{nij} - \overline{{K}_{n}} )}^{2} )}} \qquad ( 27 )$$

Wherein, Kᵢⱼ and Kₙᵢⱼ respectively represent the i-th row and j-th column elements of the global factor coordinate matrix and the two-dimensional coordinate matrix of each bivalve characteristic array column, and Kᵢⱼ and Kₙᵢⱼ respectively represent the average values of all elements in the matrices. Sort according to size of the obtained variable contribution rate, select the top five samples from the four age groups for enrichment extraction experiments to represent the microplastic accumulation situation of their age groups.

Preferably, the steps of step S4 specifically are:
S41. Pretreatment: Separately dissect tissues of individual bivalves, and perform freeze-drying;
S42. Enzyme digestion: Use trypsin solution to digest organic matter of the freeze-dried tissues to obtain an enzyme digestion solution;
S43. pH enhancement: Adjust the enzyme digestion solution to 7.5 to obtain a tissue digestion solution rich in filamentous coagulants;
S44. Hydrogen peroxide progressive digestion: Use hydrogen peroxide to completely eliminate filamentous condensate-rich cells, releasing residual microplastics in cell gaps to obtain a hydrogen peroxide digestion solution;
S45. First filter membrane purification: Perform vacuum filtration on the enzyme digestion solution and the hydrogen peroxide digestion solution, and repeat several times to obtain a microplastic purification membrane for later use;
S46. Second filter membrane purification: Extract microplastics from the purified filter membrane to remove residual organic matter on the membrane.

The freeze-drying pretreatment in Step S41 is to remove moisture from samples in the form of water vapor to retain the substances in the sample, which can reduce microplastic loss in samples, obtain the net dry weight of samples, and also improve the rate of subsequent organic matter digestion.

More preferably, the steps of the enzyme digestion in step S42 specifically are:
S421. Preparation of trypsin solution: Dissolve 6.80 g of potassium dihydrogen phosphate in 500 mL of water, then adjust pH to 7.5 with 0.1 mol/L potassium hydroxide solution, add 10.00 g of trypsin, and dilute to 1 L after dissolving in water;
S422. Enzyme digestion: Add trypsin solution to the freeze-dried bivalve tissues at the ratio of 1 g bivalve: 30 mL trypsin solution, and shake to dissolve to obtain an enzyme digestion solution with filamentous condensates.

The microplastic extraction protocol of the present application eliminates the grinding homogenization and flotation steps in previous patents, and instead uses a stage-wise pH enhancement and enzyme-hydrogen peroxide mixed digestion method to construct a microplastic extraction method completely applicable to bivalves in extreme deep-sea environments. This method uses the complete digestion capability of biochemical reactions to replace traditional physical extraction steps, completely releasing microplastics within organisms while reducing the microplastic damage effects caused by chemical reagents, while also considering future further microplastic aging test steps, to achieve non-destructive detection requirements for biological adsorption of microplastics in extreme deep-sea environments.

Preferably, the steps of step S5 specifically are:
S51. Stereomicroscope observation: Place the purified microplastic membrane obtained in step S4 under a stereomicroscope, find suspected microplastics >100 μm, record the microplastic morphological parameters, and transfer the microplastics onto a 25 mm glass fiber filter membrane;
S52. Microscope infrared identification: Select a representative suspected microplastic on the glass fiber filter membrane, perform qualitative analysis under μFTIR, and set the spectrum library match rate greater than 70%;
S53. Raman observation identification: Place the purified filter membrane after stereomicroscope observation on the Raman laser spectrometer, and observe and identify microplastics having morphological parameters of 1-20 μm on the membrane, wherein microplastics have a spectrum library match rate greater than 70%;
S54. Laser infrared identification: Use anhydrous ethanol to extract the purified filter membrane after identification by the Raman spectrometer and concentrate, add the concentrate dropwise on a cleaned high-reflective glass, select the area under the Agilent LDIR laser infrared spectrometer for component determination, and set the match rate greater than 0.7;
S55. Abundance correction: After procedural identification is completed, subtract the abundance and type of microplastics measured by the Raman spectrometer from the microplastics measured by the laser infrared spectrometer, then combine with the data measured by μFTIR to obtain the full-scale microplastic abundance of a certain tissue of the individual bivalve; wherein all tissue microplastic abundances are summed as the abundance of microplastic accumulation of the corresponding individual bivalve.

Step S5 targets microplastics of 0.1-5 mm on the membrane, which can be measured using microscopes, such as stereomicroscopes, picked out and transferred to a new membrane. Then the new membrane is placed on an infrared spectrometer to identify microplastic types and aging degree, such as a Fourier transform microscopic infrared spectrometer. The purified filter membrane after picking is observed through a Raman spectrometer for identifying 1-20 μm microplastics, then after enrichment extraction operations, 20-100 μm microplastics are identified through an infrared spectrometer, such as a laser infrared spectrometer (Agilent 8700 LDIR). These three identification process protocols achieve full coverage of all microplastic sizes, facilitating comprehensive analysis of the occurrence state of microplastics within bivalve bodies and the physical changes of microplastics after entering the deep sea.

The method proposed in the present application uses the coupling relationship between the efficiency and total amount of microplastic adsorption by bivalves of different age groups, which are indicator organisms in deep-sea methane seeps, to substitute for the microplastic uptake of the entire extreme ecosystem, to explore the long-term accumulation process of microplastics in this region, and fill the gap in the field of microplastic capture and transformation processes by seafloor ecosystems under extreme environments.

Preferably, the steps of step S6 specifically are:
S61. Drilling and crushing: Take out the preserved bivalve shells dissected in step S4, and after cleaning, find the inorganic calcium carbonate prismatic interlayer in the middle layer of the shell under the micro-Raman spectrometer, and use a micropore drill to precisely drill a sample for later use;
S62. First acid wash treatment: Clean the ground powder obtained in step S61 with dilute hydrochloric acid solution;
S63. Second alkali wash treatment: Wash the obtained acid-washed powder with NaOH solution;
S64. Third acid wash treatment: Wash the obtained alkali-washed powder again with dilute hydrochloric acid solution, then wash the residual acid washing solution and dry for later use;
S65. Thermal decomposition: Place the dried fine powder in a thermal decomposition furnace and perform thermal decomposition;
S66. Carbon dioxide capture: Absorb carbon dioxide produced during thermal decomposition by a gas capture system containing 2 M sodium hydroxide solution connected to the thermal decomposition furnace;
S67. Carbon dioxide purification: Add hydrochloric acid dropwise to release the carbon dioxide from the absorbent until no bubbles are produced, and after passing the gas through an absorption column containing solid KOH, introduce the gas into a gas chromatograph; if the mass spectrum shown by the MS detector has only a peak at m/z 44 with other peaks being absent or very small, this means that the carbon dioxide sample purification is successful, and the next step can be performed;
S68. Carbon fixation: Set the preheating temperature in a reduction furnace, place excess iron powder as catalyst, and under hydrogen gas as isolating gas, introduce the remaining purified carbon dioxide gas for reaction, and take out the fixed solid carbon after the reduction furnace cools to room temperature;
S69. Carbon-14 dating: Load the prepared pure carbon powder into a sample chamber of an isotope mass spectrometer, set the spectrometer to thermal ionization mode, set the ion source energy at 2000V, set the ion accelerator to 300 WV, and measure ion currents at specific mass/charge ratios of −14 and −12; based on the measured ion beam intensities of carbon-14 and carbon-12, I_{C-14} and I_{C-12}, set the normalization factor as k, and calculate the relative content R of carbon-14 and carbon-12, with the expression shown in equation (28):

$$R = \frac{{I}_{C - 14}}{{I}_{C - 12}} \cdot k \qquad ( 28 )$$

S610. Age estimation: Substitute the measured relative ratio of carbon-14/carbon-12 of each individual bivalve shell, the half-life of carbon-14 in the ocean, and the modern water environment carbon ratio into the formula of the carbon-14 dating method, and simultaneously use the CALIB software to correct the calculation result, and set the marine reservoir effect correction value as −178±50 yr to obtain the relatively accurate survival age of each shell.

The method proposed by the present application uses carbon-14 dating to identify the shells of indicator bivalves in seafloor extreme ecosystems that record age information, performs longitudinal accumulation curve fitting for shells of different age stages, deeply explores the microplastic accumulation of each bivalve from birth to death, assembles them into long-term microplastic accumulation curves for marine extreme ecosystems, revealing the dynamic migration and transformation process mechanisms of microplastics from production to deep-sea domains affecting marine extreme ecological environments.

More preferably, the steps of step S610 specifically are: Based on the carbon-14 dating formula, substitute the determined carbon-14/carbon-12 relative ratio in each bivalve sample, perform decay calculations based on the half-life of carbon-14 in the ocean and the modern water environment carbon ratio of approximately 1.176×10⁻¹², and calibrate the determination results using CALIB software and express as calendar age yr cal BP, with the marine reservoir effect correction value of −178±50 yr to finally obtain the relative estimated age of each shell; based on the obtained relative content R, set λ as the decay constant and R₀ as the modern carbon standard value, and substitute the data to calculate the accurate age t of the sample, with the expression shown in equation (29):

$$t = - \frac{1}{λ} ln \frac{R}{{R}_{0}} \qquad ( 29 )$$

Preferably, the steps in step S7 specifically are:
S71. Setting standards: Set the microplastic concentration in the surrounding seawater as uniform and constant under ideal conditions, set the ratio of microplastic abundance in bivalve lip tissue to environmental microplastic concentration as a constant feeding rate, set the abundance ratio of microplastics in gills and visceral mass as the absorption coefficient of bivalves, and set the inverse ratio of microplastic abundance between visceral mass and intestines as the elimination coefficient for bivalve transfer to the external environment to obtain the microplastic accumulation rate for each mussel in extreme environments;
S72. Based on the typical mussel populations selected from each age group, calculate the average microplastic accumulation rate for each age group, substitute the time points obtained from shell dating, and approximately establish a time-series model for microplastic accumulation during the entire lifecycle of individual mussels in methane seeps from birth to natural death;
S73. Extrapolate the average microplastic accumulation rates and accumulation amounts from each age group to the entire ecosystem proportionally through collection of sampling statistical data, and infer the degree of microplastic accumulation and potential pollution characteristics in the entire methane seep ecosystem.

More preferably, the steps in step S7 specifically are:

First, set the surrounding seawater microplastic concentration as uniform and constant for ideal conditions (C_{water}), set the feeding rate of individual mussels as constant (E_{bei}), set the absorption coefficient for mussels absorbing microplastics from gills to cellular tissues as constant (S_{bei}), and set the elimination coefficient for mussels transferring microplastics from tissue cells to the external environment as constant (M_{bei}), then the accumulation rate A(t) of microplastics within mussel bodies is shown in equations (30)-(31).

$$\frac{dA ( t )}{dt} = {S}_{bei} \times {C}_{water} \times {E}_{bei} - {M}_{bei} \times A ( t ) \qquad ( 30 )$$

$$A ( t ) = \frac{{S}_{bei} \times {C}_{water} \times {E}_{bei}}{{M}_{bei}} \times ( 1 - {e}^{- {M}_{bei} \times t} ) \qquad ( 31 )$$

Under steady-state conditions, when (t→t₀), the accumulation rate A(t) tends toward a stable value A(t₀). At this time, the accumulation amount at steady state can be expressed as equation (32):

$$A ( {t}_{0} ) = \frac{{S}_{bei} \times {C}_{water} \times {E}_{bei}}{{M}_{bei}} \qquad ( 32 )$$

Use the enrichment coefficient (EC) to measure the extent of microplastic accumulation within bivalve organisms relative to their concentration in the environment, then the EC calculation formula is shown in equation (33):

$$EC = \frac{{S}_{bei} \times {E}_{bei}}{{M}_{bei}} \qquad ( 33 )$$

Based on the microplastic content contained in the top five typical individual bivalves substituting for community characteristics selected from each age stage within the 4 groups and their corresponding carbon-14 estimated ages, jointly establish a time series model. Extrapolate the temporal microplastic adsorption patterns of individual bivalves obtained from all measurement data to the entire bivalve bed through sampling patterns, wherein y̅ᵢ is the average value of carbon-14 estimated ages measured for the corresponding top five bivalves within each age group, and i-juvenile, young, middle-aged, or elderly, and approximately infer the microplastic accumulation rate (vₙ) and total system microplastic accumulation (Tₙ) in the entire seafloor extreme environment ecosystem, as shown in equations (34)-(40):

$${v}_{juvenile} = \frac{{\sum }_{1}^{n} ( A ( {t}_{juvenile n} ) )}{n} \qquad ( 34 )$$

$${v}_{young} = \frac{{\sum }_{1}^{n} ( A ( {t}_{young n} ) )}{n} \qquad ( 35 )$$

$${v}_{middle - aged} = \frac{{\sum }_{1}^{n} ( A ( {t}_{middle - aged n} ) )}{n} \qquad ( 36 )$$

$${v}_{elderly} = \frac{{\sum }_{1}^{n} ( A ( {t}_{elderly n} ) )}{n} \qquad ( 37 )$$

$${v}_{1} = \frac{\begin{matrix}{v}_{juvenile} \cdot {N}_{juvenile} + {v}_{young} \cdot {N}_{young} + {v}_{middle - aged} \cdot \\ {N}_{middle - aged} + {v}_{elderly} \cdot {N}_{elderly}\end{matrix}}{{N}_{1} + {N}_{2} + {N}_{3}} \qquad ( 38 )$$

$${v}_{n} = {v}_{1} \cdot {N}_{total} \qquad ( 39 )$$

$${T}_{n} = \frac{\begin{matrix}{N}_{juvenile} \cdot {\int }_{0}^{\overline{{y}_{juvenile}}} A ( t ) + {N}_{young} \cdot {\int }_{0}^{\overline{{y}_{young}}} A ( t ) + \\ {N}_{middle - aged} \cdot {\int }_{0}^{\overline{{y}_{middle - aged}}} A ( t ) + {N}_{elderly} \cdot {\int }_{0}^{\overline{{y}_{elderly}}} A ( t )\end{matrix}}{{N}_{1} + {N}_{2} + {N}_{3}} \cdot {N}_{total} \qquad ( 40 )$$

Preferably, the bivalves include mussels, white clams, and vesicomyids.

Compared with the prior art, the present application has the following advantages:

1. Different from conventional fixed-point sampling and individual pollution research, the present application uses sampling survey methods requiring only partial bivalve groups with typical community characteristics, then through statistical testing and selection can achieve large-scale community research and analysis, greatly reducing the total number of samples needed for experiments, improving detection efficiency, saving time, manpower, and cost, and providing a basic technical means for studying the extent of ecosystem microplastic accumulation. Moreover, the present application optimizes conventional microplastic extraction experiments at multiple points, including no homogenization, using mixed digestion methods, removing flotation steps, etc., thereby improving the in-situ authenticity of microplastics and reducing microplastic loss, ensuring experimental result accuracy and comprehensiveness. Additionally, the streamlined microplastic identification protocol adopted by the present application sequentially achieves full-scale identification of microplastic sizes, and corrected data can be used to comprehensively understand the abundance and types of microplastic accumulation by this biological community. In the analysis stage, the present application first applies carbon-14 dating to the time series of seafloor bivalve biological adsorption of microplastics, and uses this combination to demonstrate the long-term dynamic microplastic adsorption capacity of methane seep ecosystems, filling the gap in this field.

2. The method proposed by the present application is simple and easy to operate, suitable for exploring characterization methods of microplastic accumulation by bivalves in deep-sea extreme ecosystems, further extrapolating to the temporal course and historical accumulation of microplastic uptake by entire extreme ecosystems, solving the problem of large-scale sampling extraction and analysis in extreme deep-sea environments. The present application can be used to systematically explore the dynamic adsorption evolution process of microplastics by deep-sea bivalves over nearly a century, aiming to enrich the theoretical framework regarding deep-sea behavior of microplastics and resulting ecological effects, providing scientific verification strategies for the impact of microplastic pollution history in specific sea areas.

### DESCRIPTION OF THE DRAWINGS

FIG. 1 is a technical flow chart of the method proposed in the present application;

FIG. 2 is a graph showing the contribution ranking of the age-body length matrix in the multivariate factor analysis;

FIG. 3 is a graph showing the accumulation of microplastics in mussels in the methane seep.

### SPECIFIC IMPLEMENTATIONS

The following embodiments are further descriptions of the present application and are not limitations on the present application.

Unless otherwise defined, all technical terms used below have the same meaning as commonly understood by those skilled in the art. The technical terms used in this document are only for the purpose of describing specific embodiments and are not intended to limit the scope of protection of the present application. Unless otherwise specified, experimental materials and reagents in this document are conventional commercially available products in the art. The number of times to sample living bivalves may be adjusted according to actual conditions. The formula proposed in the present application uses three times as an example, and other sample numbers are also within the scope of protection of the present application.

Instruments and equipment used in the following embodiments:

Vernier caliper, metal scissors, stainless steel tweezers, sampling needle, scalpel, surgical tray, spring scissors, medicine spoon, glass rod, vacuum filtration device, freeze dryer, oven, electronic analytical balance, graphite electric heating plate, pH meter, magnetic stirrer, constant temperature shaker, laser infrared spectrometer, Fourier transform microscopic infrared spectrometer, stereomicroscope, high-resolution confocal microscopic Raman laser spectrometer, remotely operated vehicle (ROV), multibeam bathymetry system, Television grab, seawater circulation system, −20° C. refrigerator, 4° C. refrigerator, micropore drill, pyrolysis furnace, gas chromatograph, reduction furnace, insulated pliers, isotope mass spectrometer.

Reagents and consumables used in the following embodiments:

Potassium hydroxide, anhydrous ethanol solution, potassium dihydrogen phosphate, trypsin, sodium chloride, concentrated hydrochloric acid, sodium hydroxide, 30% hydrogen peroxide solution, all are conventional commercial products. 0.45 μm glass fiber filter membrane, 0.22 μm aqueous micropore filter membrane, 2500 mesh steel membrane, 60 mm aluminum storage box, 10 mL glass syringe, sealing film, tin foil, 50 mL beaker, 2 L beaker, 50 mL stoppered ground-mouth conical flask, 100 mesh stainless steel sieve, glass Petri dish, 5 g glass small tube, adsorption column, Fe powder, hydrogen, self-sealing bag, ultrapure water, high-reflective glass.

Software and versions involved in the analytical methods used in the following embodiments:

Approximate area estimation method uses Office Excel for batch calculation.

Multivariate factor analysis uses R language version 4.1.2, mainly involving R packages FactoMineR, factoextra, and corrplot.

Adsorption kinetics model uses Python 3.10 with matplotlib library version 3.5.2 for calculation. Visualization uses R language version 4.1.2, involving R package ggplot2.

### Embodiment 1

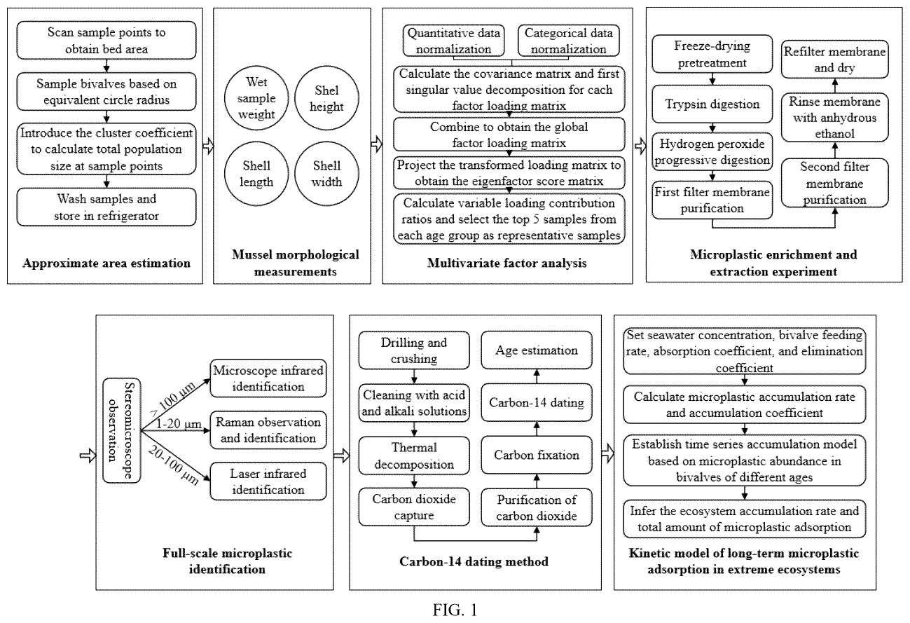

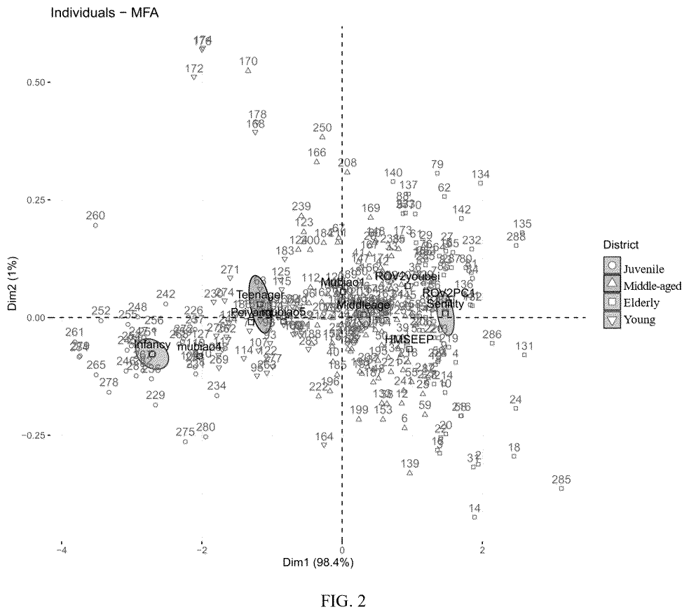

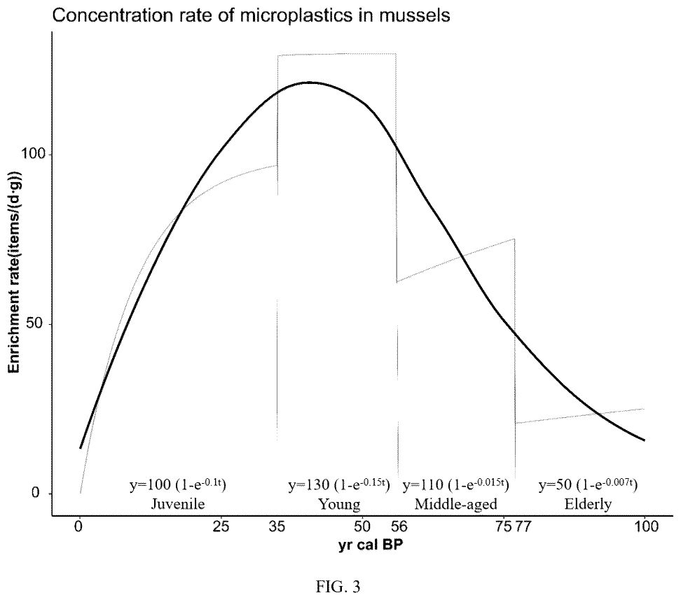

A method for large-scale detection of the degree of accumulation of microplastics by deep-sea bivalves, taking the extreme environment in the South China Sea—methane seeps—as the research environment, and selecting the indicator organism—Bathymodiolus platifrons (hereinafter referred to as “mussel”). By extracting the average abundance of microplastics accumulated by mussels of different age groups and determining their ages, a dynamic accumulation curve of microplastic adsorption within one hundred years in the seafloor methane seep ecosystem is obtained through inversion. Experimental samples were taken from mussel beds near a small plume vent in a deep-sea methane seep, as shown in FIG. 1, which includes the following steps:
S1. Collect and estimate the total number of mussels in the area.
S11. Use a remotely operated vehicle (ROV) in the deep-sea methane seeps to search for living mussel beds in the early and middle development stages of methane seeps, use a multibeam bathymetry system to measure the bed area, and find its equivalent diameter and geometric center.
S12. Lower a Television grab to sample the mussel bed three times, with the sample positions located at the two ends and the midpoint of the equivalent circle radius. After sampling and retrieval, count the number of living mussels (excluding remnant mussels), and use a cruising water device to wash off the surface mud of the mussels. After draining the water, pack the mussels separately in sealed bags and freeze them at −20° C.
S13. Based on the number of living mussels sampled three times, introduce the cluster coefficient C of living mussels to roughly estimate the total number of living mussels in the entire mussel bed.
S2. Mussel morphology measurement: Take out the sampled mussel samples from the freezer to thaw, and perform morphological data measurements and weighing in batches.
S21. Measure the three body-length parts of each mussel in parallel using a vernier caliper, and read three measurement values for each part to take the average. Measure the wet weight of the corresponding mussel using an electronic analytical balance. Record the ecological morphology data obtained, including shell length L, shell width W, shell height H, and wet weight M of each mussel.
S22. According to the different shell length-age standards of mussels (0<L<55 mm, 55 mm<L<80 mm, 80 mm<L<110 mm, L>110 mm), divide all measured mussels into four age groups: juvenile, young, middle-aged, and elderly, in order of shell length, and label the mussels of each age group.
S3. Multivariate factor analysis: Based on the obtained morphological data, associate and analyze the relationship between mussels of different ages on the seafloor and their external length and wet weight using a multifactor statistical analysis method, and identify the top five individual mussels representing the characteristics of each of the four age groups, resulting in a total of twenty mussels.
S31. Add a new age classification array to the original four columns of basic data, and replace the age group of each mussel (juvenile, young, middle-aged, elderly) with numbers 1, 2, 3, 4. Set the age groups as a categorical variable array, and set the other four columns of morphological arrays as quantitative variable arrays.
S32. Create an indicator matrix for the existing age classification array, calculate its standardized contingency table, and perform singular value decomposition. Normalize the first singular value according to the obtained diagonal matrix elements, expand the age classification array into two-dimensional space for display, and obtain the MCA standardized coordinate matrix.
S33. Perform normalization or centering standardization on the remaining four columns of quantitative arrays, calculate their covariance matrix, and obtain their eigenvalues and eigenvectors. Select the first k eigenvectors to form matrix P. Project into a new two-dimensional space to obtain the coordinate matrix after dimensionality reduction, and from this, calculate the factor score coefficient matrix of each quantitative array after dimensionality reduction.
S34. After normalization or centering, divide the four columns of quantitative arrays by the square root of the eigenvalue of the first axis of their covariance matrix to obtain the factor loading matrix describing the linear combination relationships between the four quantitative arrays and the common factors, and merge this loading matrix with the first singular value standardized matrix of the age classification array to form a global factor loading T matrix.
S35. Perform multivariate factor analysis on the global factor loading T matrix, and convert matrix TTT into projection matrix P. Project matrix T into the multivariate factor analysis model ranking chart through matrix P to calculate the variable load contribution rate of the five morphological arrays to the global factor coordinate matrix.
S36. Rank the variable load contribution rate of each array, and select the top five mussels of each age group as representatives of microplastic accumulation characteristics of the community in the corresponding age group, as shown in FIG. 2.
S4. Microplastic enrichment and extraction experiment: Conduct Microplastic enrichment and extraction experiments on tissues of the selected typical mussel groups.
S41. Freeze-drying pretreatment: Separately dissect tissues of individual mussels, and perform freeze-drying pretreatment to remove water from mussel tissues in the form of water vapor, achieving non-destructive drying of microplastics in mussel bodies.
S411. Thawing: Select required mussels from the −20° C. freezer, place them in a 4° C. refrigerator, and thaw for 1-2 h.
S412. Dissection: Use spring scissors to dissect the visceral mass, gill, lip, intestine, foot, adductor muscle, and mantle tissues of the whole individual mussel, and clean the dissected tissues. Handle the entire operation environment in a ventilated environment of a dissection table. Before dissection, soak and clean all vessels and dissection tools to be used with filtered ultrapure water. Wrap the surface of the dissection table with tin foil.
S413. Cleaning: Weigh 35.03 g of sodium chloride and add it to 1 L of water. Shake to dissolve to prepare the brine cleaning solution, and filter three times with a 0.45 μm glass fiber filter membrane. Use the brine cleaning solution to clean each dissected tissue. After three cleanings, leave the tissues to drain and place them in a 60 mm aluminum box.
S414. Weighing of wet weight: Place each aluminum box containing mussel tissue on the analytical balance, weigh each tissue's wet weight in tare mode, and record the data.
S415. Frozen storage: Place each aluminum box after weighing into a −80° C. freezer, and attach a wet weight label.
S416. Freeze drying: Loosen the lid of each sealed aluminum box slightly. When the cold trap temperature of the freeze dryer reaches −60° C., cover with a vacuum cover and start vacuum pumping. Reduce the chamber vacuum below 100 Pa, and freeze-dry each tissue under this condition for 24 h.
S417. Take each freeze-dried mussel tissue out of the freeze dryer, place it on an analytical balance with an accuracy of 0.1 mg, and weigh it. Obtain and record the dry weights of the corresponding tissues of gill, mantle, adductor muscle, foot, byssus, visceral mass, and intestine.
S42. Trypsin digestion: Use trypsin solution to digest organic matter of the freeze-dried mussel tissues.
S421. Preparation of trypsin solution: Weigh 6.80 g of potassium dihydrogen phosphate, add 500 mL of water, shake to dissolve, then adjust pH to 7.5 with 0.1 mol/L potassium hydroxide solution. Add 10.00 g of trypsin, and after dissolving in water, dilute to 1 L, and filter three times with a 0.45 μm glass fiber filter membrane.
S422. Enzyme digestion: Place each freeze-dried tissue into a 50 mL stoppered ground-mouth conical flask, and add trypsin solution at the ratio of 1 g mussel: 30 mL digestion solution. Place the tissue digestion solution in a constant temperature shaker at 37° C. and shake at 100 rpm and digest for 24 h, taking out every 3 h to place under a magnetic stirrer to accelerate shaking, thereby obtaining enzyme digestion solution with many filamentous condensates.
S43. Hydrogen peroxide progressive digestion: Use a progressive digestion method to completely eliminate filamentous condensate-rich cell groups, and release residual microplastics in cell gaps.
S431. Preparation of pH adjustment solution: Weigh 0.68 g of potassium dihydrogen phosphate, add 50 mL of water to dissolve, filter three times with a 0.45 μm glass fiber filter membrane, and shake. Weigh 2.81 g of potassium hydroxide, add 50 mL of water to dissolve, filter three times with a 0.45 μm glass fiber filter membrane, and shake. Bottle the prepared pH adjustment solution for use.
S432. Solution pH adjustment: After 24 h enzyme digestion, take out the conical flask containing the trypsin digestion solution, measure acidity with a pH meter, use a cleaned 10 mL glass syringe to take the pH adjustment solution, maintain the pH value of the enzyme digestion solution at 7.5, and use excess enzyme digestion solution to clean the pH meter. After adjustment, place the flask back into the constant temperature shaker, and repeat this operation every 12 h a total of four times.
S433. Hydrogen peroxide digestion: Use tweezers to pick filamentous condensate from the conical flask into a new conical flask, add 30 mL of 30% hydrogen peroxide digestion solution (GR), and clean the tweezers. Digest residual cell adhesive substances in the sample at 60° C. on a graphite electric heating plate for 3 h, until no bubbles are produced in the digestion solution, and digestion is completed.
S44. First filter membrane purification: Add the enzyme digestion solution and the hydrogen peroxide digestion solution together into a filtration flask, perform vacuum filtration, and repeat the filtration three times. Then rinse the inner wall of the filter with ultrapure water, and place the purified filter membrane into a 60 mm aluminum box for storage and labeling.

Wherein, the filter membrane used is a 2500 mesh steel membrane in order to avoid interference caused by a traditional glass fiber membrane, and the steel membrane also improves the effect of subsequent infrared spectroscopy identification of microplastics.
S45. Second filter membrane purification: Use anhydrous ethanol solution to extract microplastics from the filter membrane and remove residual organic matter.
S451. Place the purified filter membrane from the first treatment upright in a 50 mL beaker, add about 15 mL of anhydrous ethanol solution until the membrane surface is completely covered, seal the beaker with cleaned tin foil fixed with a rubber band, place the beaker on a shaker at 90 rpm, and shake the beaker for 12 h to completely release microplastic particles into solution.
S452. Take out the filter membrane after ethanol extraction, and repeatedly wash the filter membrane with anhydrous ethanol until no obvious particles remain.
S453. Filtration and membrane preparation: Vacuum-filter the anhydrous ethanol cleaning solution again, filter three times, and lastly repeatedly clean the beaker wall and carrier with anhydrous ethanol to enrich all microplastics onto the filter membrane. Place the obtained filter membrane after the second purification into a 60 mm aluminum box, and dry it in an oven at 60° C. for 4 h.
S5. Full-scale microplastic identification: Use a procedural protocol to identify microplastics of 0.001-5 mm on the membrane in batches, to obtain corresponding type, morphology, size, and color information.
S51. Stereomicroscope observation (>100 μm): Place the dried filter membrane after second purification under a stereomicroscope, observe the surface of the stainless steel membrane in a “Z” pattern, use the measuring ruler tool to find suspected microplastics >100 μm, record the morphological parameters, and transfer with a sampling needle onto a 25 mm glass fiber filter membrane.
S52. Microscope infrared identification (>100 μm): Select a representative suspected microplastic on the glass fiber filter membrane, perform qualitative analysis under μFTIR, and set the spectrum library match rate greater than 70%.
S53. Raman observation identification (1-20 μm): Place the purified filter membrane after stereomicroscope observation on the stage of a high-resolution confocal microscopic Raman laser spectrometer, scan in a “Z” pattern, and observe and identify microplastics having morphological parameters of 1-20 μm on the membrane. Microplastics are identified having a spectrum library match rate greater than 70%.
S54. Laser infrared identification (20-100 μm): Use anhydrous ethanol again to extract the purified filter membrane after identification by the Raman spectrometer, and concentrate into a 100 μL suspension. Add the suspension dropwise onto cleaned high-reflective glass, select the area under the Agilent LDIR laser infrared spectrometer, and determine the components with a match rate set above 0.7.
S55. Abundance correction: After the procedural identification is completed, subtract the abundance and type of microplastics measured by the Raman spectrometer from the microplastics measured by the laser infrared spectrometer, then combine with the data measured by μFTIR to obtain the full-scale microplastic abundance of a certain tissue of the individual mussel. Sum all tissue microplastic abundances as the abundance of microplastic accumulation of the corresponding individual mussel.
S6. Carbon-14 dating method: Use carbon-14 dating to determine the survival time points of the corresponding individual mussels.
S61. Drilling and crushing: Take out the mussel shells preserved in ultrapure water during the previous dissection and clean them with ultrapure water to remove contaminants. Under the micro-Raman spectrometer, find the inorganic calcium carbonate prismatic interlayer in the middle layer of the shell, use a micropore drill to precisely drill about 1 g of sample, and place the sample in a small glass tube for later use.
S62. First acid wash treatment: Clean the ground fine powder with 1 M dilute hydrochloric acid solution under a 100 mesh stainless steel sieve, and slightly etch the shell powder surface to remove possible secondary calcium carbonate.
S63. Second alkali wash treatment: Wash the acid-washed powder obtained in the previous step with 1 M NaOH solution to remove most of the remaining organic impurities.
S64. Third acid wash treatment: Wash the obtained alkali-washed powder again with 1 M dilute hydrochloric acid solution, then wash the residual acid washing solution with ultrapure water, and dry the powder in an oven at 70° for 12 h to ensure no impurities are introduced in the oven to interfere with the carbon component of the fine powder.
S65. Thermal decomposition: Take 200 mg of the dried fine powder and place it in a thermal decomposition furnace, with the parameters set at 850° C., 10° C./min heating rate, 2 h temperature holding time, and 3 h cooling time.
S66. Carbon dioxide capture: Absorb carbon dioxide produced during thermal decomposition by a gas capture system containing 2 M sodium hydroxide solution connected to the thermal decomposition furnace.
S67. Carbon dioxide purification: Add 5 M hydrochloric acid dropwise to release the carbon dioxide from the absorbent until no bubbles are produced, and after passing the gas through an absorption column containing solid KOH, introduce 1 mL of gas into a gas chromatograph. If the mass spectrum shown by the MS detector has only a peak at m/z 44 with other peaks being absent or very small, this means that the carbon dioxide sample purification is successful, and the next step is performed.
S68. Carbon fixation: In a reduction furnace set to 800° C. and preheated for 2 h, place excess iron powder as catalyst, and under hydrogen gas as isolating gas, introduce about 60 mL of the remaining purified carbon dioxide gas. Set a reaction time of 2 h, and after the reduction furnace cools to room temperature, take out the fixed solid carbon by pliers.
S69. Carbon-14 dating: Load the prepared pure carbon powder into a sample chamber of an isotope mass spectrometer, set the spectrometer to thermal ionization mode, set the ion source energy at 2000V, set the ion accelerator to 300 WV, and measure ion currents at specific mass/charge ratios of −14 and −12, to obtain the content and relative ratio of carbon-14 to carbon-12 in the shell sample.
S610. Age estimation: Substitute the measured relative ratio of carbon-14/carbon-12 of each individual mussel shell, the half-life of carbon-14 in the ocean (about 5568 years), and the modern water environment carbon ratio of about 1.176×10⁻¹² into the formula of the carbon-14 dating method. Use the common method (CALIB software) recommended by the International 14C Committee to correct the calculation result, and set the marine reservoir effect correction value as −178±50 yr to obtain the relatively accurate survival age of each shell.
S7. Kinetic model of long-period adsorption of microplastics in extreme ecosystems: Calibrate environmental conditions, and combine the adsorption kinetic model according to the abundance of microplastic accumulation in the separate mussel tissues to construct a microplastic accumulation curve in the mussel bodies. By combining the survival age of each mussel with sampling collection patterns, the microplastic accumulation rate and accumulation amount is reverse-deduced.
S71. Setting standards: Set the microplastic concentration in the surrounding seawater as uniform and constant under ideal conditions, set the ratio of microplastic abundance in mussel lip tissue to environmental microplastic concentration as a constant feeding rate, set the abundance ratio of microplastics in gills and visceral mass as the absorption coefficient of mussels, and set the inverse ratio of microplastic abundance between visceral mass and intestines as the elimination coefficient for mussel transfer to the external environment. Using the formula, the microplastic accumulation rate for each mussel in extreme environments can be calculated.
S72. Based on the typical mussel populations selected from each age group, calculate the average microplastic accumulation rate for each age group, substitute the time points obtained from shell dating, and approximately establish a time-series model for microplastic accumulation during the entire lifecycle of individual mussels in methane seeps from birth to natural death, as shown in FIG. 3.
S73. Extrapolate the average microplastic accumulation rates and accumulation amounts from each age group to the entire ecosystem proportionally through collection of sampling statistical data, and infer the degree of microplastic accumulation and potential pollution characteristics in the entire methane seep ecosystem over the past century.

Experimental data related to the aforementioned ecosystem accumulation of microplastics is shown in Table 1.

TABLE 1

| | | | | | | Microplastic | | | | | | |
|---|---|---|---|---|---|---|---|---|---|---|---|---|
| | | Shell | Shell | Shell | Wet | abundance | Carbon-14 | ν_{juvenile/young/n} | | ν₁ | νₙ | Tₙ |
| MFA | Age | length/ | width/ | height/ | weight/ | items/ | dating/ | items/ | Nₜₒₜₐₗ/ | items/ | items/ | items/ |
| order | group | mm | mm | mm | g | g | yr | yr/g | individual | yr/g | yr/g | g |
| 1 | Juvenile | 35.8 | 14.7 | 21.3 | 5.21 | 986 | 25 ± 3 | 37.3-47.5 | 88236 | 13.61 | 1200891.96 | 78057977.4 |
| 2 | | 40.9 | 17.3 | 23.4 | 7.28 | 1084 | | | | | | |
| 3 | | 44.9 | 18 | 26.2 | 7.44 | 1159 | | | | | | |
| 4 | | 34.8 | 14.4 | 19.4 | 3.48 | 956 | | | | | | |
| 5 | | 39.3 | 14.1 | 26.1 | 4.39 | 1037 | | | | | | |
| 1 | Young | 64.3 | 29.6 | 36. | 23.35 | 765 | 50 ± 3 | 15.9-17.9 | | | | |
| 2 | | 68.9 | 29.4 | 38.9 | 25.97 | 866 | | | | | | |
| 3 | | 74 | 28.6 | 40.7 | 29.42 | 782 | | | | | | |
| 4 | | 68.1 | 29.2 | 36.7 | 24.7 | 973 | | | | | | |
| 5 | | 63.26 | 27.63 | 33.54 | 22.39 | 841 | | | | | | |
| 1 | Middle- | 93.43 | 38.5 | 51.06 | 62.02 | 512 | 75 ± 4 | 7.1-7.9 | | | | |
| 2 | aged | 86.29 | 35.75 | 43.81 | 58.52 | 588 | | | | | | |
| 3 | | 90.88 | 41.78 | 49.23 | 86.43 | 579 | | | | | | |
| 4 | | 82.94 | 35.7 | 44.98 | 49.5 | 522 | | | | | | |
| 5 | | 87.37 | 38.99 | 42.65 | 55.57 | 612 | | | | | | |
| 1 | Elderly | 111.89 | 41.96 | 50.82 | 103.65 | 319 | 100 ± 7 | 3.0-3.1 | | | | |
| 2 | | 113.64 | 49.39 | 52.43 | 121.48 | 272 | | | | | | |
| 3 | | 126.04 | 54.21 | 60.57 | 138.76 | 266 | | | | | | |
| 4 | | 119.3 | 49.53 | 60.51 | 105.6 | 378 | | | | | | |
| 5 | | 126.11 | 52.34 | 61.05 | 135.84 | 289 | | | | | | |

The prior art has not extensively explored the spatial accumulation capacity of microplastics in extreme environments. There is only one remote sensing-based identification method for spatial distribution of microplastic pollution in water bodies, which can identify spatial distribution characteristics of microplastics, but does not involve the temporal accumulation capacity manifestation of ecosystems. It mainly includes the following steps:
(1) Water sampling: Collect water samples in target waters, record GPS coordinates of sampling points, filter samples with nets, and transfer residues on the filter to glass bottles;
(2) Pretreatment: Use hydrogen peroxide solution to digest natural organic matter in the water samples, use a glass fiber filter membrane for vacuum filtration to enrich microplastics onto the membrane;
(3) Identification and statistics: Use a stereomicroscope for visual inspection to qualitatively judge microplastics on the membrane, and record color, quantity, size, and shape;
(4) Remote sensing database construction: Set circular areas with a radius of 100 m centered on each sampling point as research areas, extract remote sensing spectral curve characteristics of each research area, correlate previously identified microplastic information with spectral characteristic curves, and establish a spectral library for microplastic-polluted waters;
(5) Pollution identification: Obtain hyperspectral data of unknown waters, match their spectral characteristic curves with the database for identification, rapidly achieving identification of surface water microplastic pollution areas.

Compared with Embodiment 1, the prior art only involves spatial distribution identification or pollution assessment of microplastics in shallow waters, and does not involve the dynamic rate characterization of microplastic adsorption by extreme ecosystems. Furthermore, most studies focus on immediate microplastic spatial pollution, with few strategies for expressing temporal progressive pollution or accumulation capacity of microplastics. To address gaps in the prior art, distinguishing from conventional water bodies or sediment carrier media, the present application provides a novel technical solution for expressing the degree of spatiotemporal accumulation of microplastics by using indicator organisms from extreme ecosystems as research subjects, extracting microplastics from their tissues, and developing adsorption curves of biological temporal accumulation rates of microplastics through carbon-14 dating, thereby applying statistical principles to extrapolate to accumulation patterns of microplastics in seafloor extreme ecosystems, to assess spatiotemporal distribution and accumulation trends of microplastics in special environments.

The description of the above embodiments is only for helping to understand the technical solutions of the present application and its core ideas. It should be pointed out that, for those of ordinary skill in the technical field, without departing from the principles of the present application, improvements and modifications can also be made to the present application, and these improvements and modifications also fall within the protection scope of the claims of the present application.

1. A method for measuring an extent of microplastic bioaccumulation in bivalves in extreme deep-sea environments, comprising the following steps:
S1, collecting and estimating a total number of bivalves in an area:
S11, searching for a living bivalve bed in early and middle development stages of methane seeps in deep-sea methane seeps, measuring a bed area, and finding an equivalent diameter and a geometric center of the bed area;
S12, sampling the bivalve bed several times, with sample positions located at two ends and a midpoint of an equivalent circle radius; after sampling and retrieval, counting a number of living bivalves, washing the bivalves, and storing the bivalves frozen after draining water;
S13, based on a number of bivalves sampled several times, roughly estimating a total number of living bivalves in the entire bivalve bed;
S2, bivalve morphology measurement: thawing sampled bivalve samples, performing morphological data measurements and weighing in batches, and dividing the bivalves according to increasing shell length into four age groups of juvenile, young, middle-aged, and elderly;
S3, multivariate factor analysis: based on obtained morphological data in step S2, associating and analyzing a relationship between bivalves of different ages on seafloor and their external length and wet weight using a multifactor statistical analysis method, and identifying top five individual bivalves representing characteristics of each of the four age groups, resulting in a total of twenty bivalves;
S4, microplastic enrichment and extraction: separating and extracting microplastics from each tissue of representative bivalve samples of the four age groups selected by the multivariate factor analysis in step S3, ultimately obtaining a purified microplastic filter membrane for convenient subsequent observation and detection;
S5, instrument identification: sequentially identifying microplastics with sizes ranging from 0.001-5 mm in the purified microplastic filter membrane obtained in step S4, and comprehensively analyzing an occurrence state of microplastics within bivalve bodies and physical changes of the microplastics after entering deep sea;
S6, carbon-14 dating method: using carbon-14 dating to determine chronological ages of corresponding individual bivalves, thereby converting a lifespan duration of the individual bivalves from the four age groups into time periods of microplastic absorption by seafloor bivalves;
S7, dynamic microplastic accumulation model for bivalves: using an adsorption kinetic model to construct microplastic accumulation curves within bivalve bodies; calculating accumulation coefficients to represent a degree of microplastic accumulation in sample bodies relative to an environmental concentration; based on a microplastic concentration in the top five individual bivalves representing the group characteristics selected from the four age groups and their corresponding carbon-14 estimated ages, jointly establishing a time-series model;
extrapolating temporal microplastic adsorption patterns of the individual bivalves obtained from all measurement data to the entire bivalve bed through sampling patterns, and approximately deducing a rate of microplastic accumulation and total microplastic accumulation in an entire seafloor extreme environment ecosystem.

2. The method according to claim 1, wherein the steps of step S2 specifically are: performing morphological measurements and weighing in batches for all of the bivalve samples sampled several times from the seafloor; measuring three body length dimensions of the bivalves to obtain a shell length L, shell width W, and shell height H for each living bivalve; based on general body length standards corresponding to different age groups of bivalves, establishing four shell length ranges, which are 0<L<55 mm, 55 mm<L<80 mm, 80 mm<L<110 mm, and 110 mm<L, corresponding to the four age groups classified as juvenile, young, middle-aged, and elderly.

3. The method according to claim 1, wherein the steps of step S3 specifically are:
S31, based on the morphological data obtained in step S2, performing categorical sampling, adding a new age classification array to original four columns of basic data, replacing the age groups of juvenile, young, middle-aged, elderly with numbers 1, 2, 3, 4, setting the age groups as a categorical variable array, and setting the other four columns of morphological arrays as quantitative variable arrays;
S32, creating an indicator matrix for the existing age classification array, calculating its standardized contingency table, and performing singular value decomposition; normalizing a first singular value according to obtained diagonal matrix elements, and expanding the age classification array into a two-dimensional space for display to obtain a multiple correspondence analysis (MCA) standardized coordinate matrix;
S33, performing normalization or centering standardization on the remaining four columns of quantitative arrays, calculating their covariance matrix, and obtaining their eigenvalues and eigenvectors; selecting first k eigenvectors to form matrix P, and projecting into a new two-dimensional space to obtain the coordinate matrix after dimensionality reduction, and from this, calculating a factor score coefficient matrix of each quantitative array after dimensionality reduction;
S34, after being normalized or centered, dividing the four columns of quantitative arrays by a square root of the eigenvalue of a first axis of their covariance matrix to obtain a factor loading matrix describing linear combination relationships between the four quantitative arrays and common factors, and merging this loading matrix with a first singular value standardized matrix of the age classification array to form a global factor loading matrix T;
S35, performing the multivariate factor analysis on the global factor loading matrix T, and converting matrix TTT into projection matrix P; projecting matrix T into a multivariate factor analysis model ranking chart through matrix P to calculate a variable load contribution rate of the five morphological arrays to a global factor coordinate matrix;
S36, ranking the variable load contribution rate of each array, and selecting the top five bivalve of each age group as representatives of microplastic accumulation characteristics of a community in the corresponding age group.

4. The method according to claim 1, wherein the steps of step S4 specifically are:
S41, pretreatment: separately dissecting tissues of individual bivalves, and performing freeze-drying;
S42, enzyme digestion: using trypsin solution to digest organic matter of freeze-dried tissues to obtain an enzyme digestion solution;
S43, pH enhancement: adjusting the enzyme digestion solution to 7.5 to obtain a tissue digestion solution rich in filamentous coagulants;
S44, hydrogen peroxide progressive digestion: using hydrogen peroxide to completely eliminate filamentous condensate-rich cells, releasing residual microplastics in cell gaps to obtain a hydrogen peroxide digestion solution;
S45, first filter membrane purification: performing vacuum filtration on the enzyme digestion solution and the hydrogen peroxide digestion solution, and repeating several times to obtain a microplastic purification membrane for later use;
S46, second filter membrane purification: extracting microplastics from the purified filter membrane to remove residual organic matter on the membrane.

5. The method according to claim 4, wherein the steps of the enzyme digestion in step S42 specifically are:
S421, preparation of trypsin solution: dissolving 6.80 g of potassium dihydrogen phosphate in 500 mL of water, then adjusting pH to 7.5 with 0.1 mol/L potassium hydroxide solution, adding 10.00 g of trypsin, and diluting to 1 L after dissolving in water;
S422, enzyme digestion: adding trypsin solution to the freeze-dried bivalve tissues at a ratio of 1 g bivalve: 30 mL trypsin solution, and shaking to dissolve to obtain the enzyme digestion solution with filamentous condensates.

6. The method according to claim 1, wherein the steps of step S5 specifically are:
S51, stereomicroscope observation: placing the purified microplastic filter membrane obtained in step S4 under a stereomicroscope, finding suspected microplastics >100 μm, recording microplastic morphological parameters, and transfering the microplastics onto a 25 mm glass fiber filter membrane;
S52, microscope infrared identification: selecting a representative suspected microplastic on the glass fiber filter membrane, performing qualitative analysis under μFTIR, and setting a spectrum library match rate greater than 70%;
S53, Raman observation identification: placing the purified filter membrane after stereomicroscope observation on a Raman spectrometer, and observing and identifying microplastics having morphological parameters of 1-20 μm on the membrane, wherein microplastics have a spectrum library match rate greater than 70%;
S54, laser infrared identification: using anhydrous ethanol to extract the purified filter membrane after identification by the Raman spectrometer and concentrating, adding a concentrate dropwise on a cleaned high-reflective glass, selecting an area under an Agilent LDIR laser infrared spectrometer for component determination, and setting a match rate greater than 0.7;
S55, abundance correction: after procedural identification is completed, subtracting abundance and type of microplastics measured by the Raman spectrometer from the microplastics measured by the laser infrared spectrometer, then combining with data measured by μFTIR to obtain full-scale microplastic abundance of a certain tissue of the individual bivalve; wherein all tissue microplastic abundances are summed as abundance of microplastic accumulation of the corresponding individual bivalve.

7. The method according to claim 1, wherein the steps of step S6 specifically are:
S61, drilling and crushing: taking out preserved bivalve shells dissected in step S4, and after cleaning, finding an inorganic calcium carbonate prismatic interlayer in a middle layer of the shell under a micro-Raman spectrometer, and using a micropore drill to precisely drill a sample, and the sample is placed in a small glass tube for later use;
S62, first acid wash treatment: cleaning a ground powder obtained in step S61 with dilute hydrochloric acid solution;
S63, second alkali wash treatment: washing obtained acid-washed powder with NaOH solution;
S64, third acid wash treatment: washing obtained alkali-washed powder again with dilute hydrochloric acid solution, then washing residual acid washing solution and drying for later use;
S65, thermal decomposition: placing a dried fine powder in a thermal decomposition furnace and perform thermal decomposition;
S66, carbon dioxide capture: absorbing carbon dioxide produced during thermal decomposition by a gas capture system containing 2 M sodium hydroxide solution connected to the thermal decomposition furnace;
S67, carbon dioxide purification: adding hydrochloric acid dropwise to release the carbon dioxide from an absorbent until no bubbles are produced, and after passing a gas through an absorption column containing solid KOH, introducing the gas into a gas chromatograph; if a mass spectrum shown by an MS detector has only a peak at m/z 44 with other peaks being absent or very small, this means that carbon dioxide sample purification is successful, and the next step can be performed;
S68, carbon fixation: setting a preheating temperature in a reduction furnace, placing excess iron powder as catalyst, and under hydrogen gas as isolating gas, introducing a remaining purified carbon dioxide gas for reaction, and taking out a fixed solid carbon after the reduction furnace cools to room temperature;
S69, carbon-14 dating: loading a prepared pure carbon powder into a sample chamber of an isotope mass spectrometer, setting the spectrometer to thermal ionization mode, setting an ion source energy at 2000V, setting an ion accelerator to 300 WV, and measuring ion currents at specific mass/charge ratios of −14 and −12; based on measured ion beam intensities of carbon-14 and carbon-12, I_{C-14} and I_{C-12}, setting a normalization factor as k, and calculating a relative content R of carbon-14 and carbon-12, with an expression shown in equation (28):
$$R = \frac{{I}_{C - 14}}{{I}_{C - 12}} \cdot k \qquad ( 28 )$$
S610, age estimation: substituting a measured relative ratio of carbon-14/carbon-12 of each individual bivalve shell, a half-life of carbon-14 in an ocean, and a modern water environment carbon ratio into a formula of the carbon-14 dating method, and simultaneously using a CALIB software to correct a calculation result, and setting a marine reservoir effect correction value as −178±50 yr to obtain a relatively accurate survival age of each shell.

8. The method according to claim 7, wherein the steps of step S610 specifically are:
based on the carbon-14 dating formula, substituting the determined carbon-14/carbon-12 relative ratio in each bivalve sample, performing decay calculations based on the half-life of carbon-14 in the ocean and the modern water environment carbon ratio of approximately 1.176×10⁻¹², and calibrating determination results using the CALIB software and expressing as calendar age yr cal BP, with the marine reservoir effect correction value of −178±50 yr to finally obtain a relative estimated age of each shell; based on the obtained relative content R, set λ as a decay constant and R₀ as a modern carbon standard value, and substituting data to calculate an accurate age t of the sample, with an expression shown in equation (29):
$$t = - \frac{1}{λ} ln \frac{R}{{R}_{0}} . \qquad ( 29 )$$

9. The method according to claim 1, wherein the steps in step S7 specifically are:
S71, setting standards: setting a microplastic concentration in surrounding seawater as uniform and constant under ideal conditions, setting a ratio of microplastic abundance in mussel lip tissue to environmental microplastic concentration as a constant feeding rate, setting an abundance ratio of microplastics in gills and visceral mass as an absorption coefficient of bivalves, and setting an inverse ratio of microplastic abundance between visceral mass and intestines as an elimination coefficient for bivalve transfer to an external environment to obtain a microplastic accumulation rate for each mussel in extreme environments;
S72, based on typical mussel populations selected from each age group, calculating an average microplastic accumulation rate for each age group, substituting time points obtained from shell dating, and approximately establishing a time-series model for microplastic accumulation during an entire lifecycle of individual mussels in methane seeps from birth to natural death;
S73, extrapolating the average microplastic accumulation rates and accumulation amounts from each age group to an entire ecosystem proportionally through collection of sampling statistical data, and inferring a degree of microplastic accumulation and potential pollution characteristics in an entire methane seep ecosystem.

10. The method according to claim 1, wherein the bivalves comprise mussels, white clams, and vesicomyids.
